(19) United States
(12) Patent Application Publication
(10) Pub. No.: US 2007/0049559 A1
(43) Pub. Date: Mar. 1, 2007

(54) Preparations comprising arylazine substituted with a carbonylic moiety to increase the activity of Gelatinase A in ocular cells

(76) Inventors: Bruce Pfeffer, Fairport, NY (US); Rosemarie Flick, Collegeville, PA (US); Stephen Bartels, Pittsford, NY (US)

Correspondence Address: Bausch & Lomb Incorporated, One Bausch & Lomb Place, Rochester, NY, 14604-2701, US

(21) Appl. No.: 11/476,496

(22) Filed: Jun. 28, 2006

Related U.S. Application Data
(60) Provisional application No. 60/694,726, filed on Jun. 28, 2005.

(51) Int. Cl. A61K 31/655; A61K 31/517; A61K 31/498; A61K 31/47

(52) U.S. Cl. 514/150; 514/249; 514/266.1; 514/310; 514/311; 514/248

(57) ABSTRACT

Preparations for controlling intraocular pressure in the eye comprise as an active compound, arylazine substituted with a carbonylic moiety, which compound is capable of effecting a “pharmacological trabeculocanalotomy” in an eye by means of reducing juxtacanalicular meshwork to promote outflow of aqueous. The organic active compound increases Gelatinase A activity in ocular cells by increasing cell membrane expression of membrane-type matrix metalloproteinases (MT-MMPs) to increase aqueous outflow as a treatment for glaucoma, e.g., primary open angle glaucoma.

### CROSS REFERENCE

This application claims the benefit of Provisional Patent Application No. 60/694,726 filed Jun. 28, 2005 and is incorporated herein by reference.

### FIELD

The present invention relates generally to small organic molecules (e.g., less than about 1000 MW, say, less than about 500 MW) capable of effecting a “pharmacologic trabeculocanalotomy” in an eye by means of reducing juxtacanalicular meshwork as a barrier to outflow of aqueous. More specifically, the present invention relates to small organic molecules that increase Gelatinase A activity in ocular cells by increasing cell membrane expression of membrane-type matrix metalloproteinases (MT-MMPs) to increase aqueous outflow as a treatment for primary open angle glaucoma. The present invention further includes methods of manufacturing and using such small organic molecules in the treatment of primary open angle glaucoma.

### BACKGROUND

The extracellular matrix (ECM) of a tissue, e.g., that of an eye, is an association of specialized proteins, glycoproteins, and proteoglycans, that impart structure to the physiological functions of connective tissues. At the cellular level, the ECM not only provides structure, flexibility and support, but also acts as a filtration barrier, mediates cell attachment and influences tissue morphogenesis and differentiation. Part of the normal functioning of the ECM involves the ECM's tightly regulated turnover, which balances the degradation and disposal of effete molecules with the secretion and integration of the various newly synthesized ECM elements.

Specialized extracellular proteolytic enzymes, termed matrix metalloproteinases (MMPs), are produced by many cell types. MMPs play an important role in the initial degradation of ECM molecules such as collagen, fibronectin and various proteoglycans. MMP activity is regulated in part through secretion as inactive proenzymes and activation by proteolytic processing to smaller molecular weight forms. This regulated activity of MMPs usually requires protease activity as well as autolytic mechanisms. MMPs are inhibited by endogenous tissue inhibitors of matrix metalloproteinases (TIMPs). For the MMP, Gelatinase A (GelA) (MMP-2; 72 kD gelatinase; type IV collagenase; E.C. 3.4.24.24), the specific proteolytic activator is known to be another member of the MMP family, namely MT-MMP. In contrast to other MMPs, MT-MMP is predominantly expressed as an integral membrane protein. There are six known subtypes of MT-MMP, hence the designations MT1-MMP for MMP-14, MT2-MMP for MMP-15, MT3-MMP for MMP-16, MT4-MMP for MMP-17, MT5-MMP for MMP-24 and MT6-MMP for MMP-25. All subtypes of MT-MMP, except for MT4-MMP, effect a cleavage in 72 kD proGelA to initiate a proteolytic “cascade” to 66 kD (intermediate), 59 kD (active) and 43 kD (“mini”) forms of GelA. MT-MMP is capable of activating GelA that is complexed with a specific inhibitory protein, TIMP-2. This places MT-MMP expression and/or activity as a major control point in the regulation of ECM turnover.

Trabecular meshwork (TM) is the tissue located at the irido-corneal angle of an eye's anterior segment. The TM is where the aqueous secreted by the ciliary epithelium flows out of the eye. The cells of the TM reside either on collagenous beams, or trabeculae, or embedded in the ECM associated with the canal of Schlemm. The canal of Schlemm is an endothelium-lined channel into which the aqueous drains. The intraocular fluid pressure (IOP) is maintained through a balance of the secretion and outflow of aqueous. Normal IOP is slightly above venous pressure, in part resulting from outflow resistance at the TM. TM outflow resistance is believed to be the result of the hydrodynamic properties of the ECM macromolecules of the TM and the ECM associated with the trabeculae.

Potentially blinding eye diseases include those termed primary open angle glaucoma (POAG) which are characterized by an insidious, progressive increase in IOP. The disease may be caused by a dysfunction in the regulation of ECM turnover at the level of the TM. There is a biochemical lesion localized to the TM, which manifests as a general excess of ECM or as an imbalance with respect to specific components of the ECM, either of which impairs the ability of fluid to leave the eye at its normal physiological rate. It has been proposed that pharmacological intervention to reduce accumulated ECM could result in a lowering of the elevated IOP characteristic of these diseases. Moreover, treatment with IOP-reducing drugs is believed to be therapeutic even in those instances where intraocular pressure appears normal, e.g., normotensive glaucoma.

A therapeutic small organic molecule capable of increasing MT-MMP expression has been decribed by Ito et al. (Ito et al., Eur. J. Biochem. 251, 353-358 (1998)). As described by Ito et al., a trifluoperazine treatment of human cervical fibroblasts resulted in MT1-MMP-induced activation of Gelatinase A (GelA). Trifluoperazine had previously been categorized as a therapeutic “antipsychotic.” Ito et al. however, classified trifluoperazine as a calmodulin antagonist, and made a similar claim for another calmodulin inhibitor, W-7, although the effect from the later compound was not particularly pronounced. Ito et al. deduced that calmodulin negatively regulates MT-MMP expression.

Using Western blot immunochemistry, the presence of MT-MMP has been documented in human ocular tissues other than TM cells, as well as in fresh and cultured porcine TM cells. (Alexander, J. and Acott, T. S., Invest. Ophthalmol. Vis. Sci. 40 (ARVO Abstracts): S506, #2670 (1999)). (Smine, A. and Plantner, J. J., Curr. Eye Res. 16:925 (1997)). In neither case was activation of GelA documented, although Alexander and Acott described increased expression of MT1-MMP with phorbol ester (phorbol 12-myristate 13-acetate). Phorbol ester, however, does not have therapeutic usefulness since it is a known carcinogen.

In addition to trifluoperazine and phorbol ester, the following agents have been shown to increase MT-MMP expression and/or GelA activation in cells other than those of the TM: Concanavalin A, interleukin-1α, orthovanadate, a hexapeptide derived from elastin, cytochalasin D, monensin, tumor necrosis factor-alpha, bacterial lipopolysaccharide, hydrogen peroxide, oxidized low density lipoproteins, hepatocyte growth factor/scatter factor, beta-amyloid peptide, activated Protein C, growth hormone, Interleukin 8, glycyl-L-histidyl-L-lysine-Cu²⁺, and lysophosphatidic acid.

U.S. Pat. No. 5,260,059, covers an agent that increases the activity of matrix metalloproteinases (MMPs). The '059 patent discloses a method of treating glaucoma by providing TM cells with an array of macromolecules, including matrix metalloproteinase-1 (MMP-1), matrix metalloproteinase-2 (MMP-2) and matrix metalloproteinase-3 (MMP-3). The class of MMPs designated as MT-MMPs had not been characterized at the time the '059 patent was filed. At the time of filing the '059 patent, physiologic activation of GelA was suspected of being brought about by means of autocatalytic mechanisms alone. Other molecules specifically mentioned in the '059 patent are basic heparin-binding growth factor, nerve growth factor, interleukin-1, interleukin-6, phorbol ester, calcium ions, zinc ions, plasmin, trypsin, and aminophenyl mercuric acetate (APMA).

US 2004/0068017 A1 covers an agent that increases Gelatinase A activity in ocular cells by reducing juxtacanalicular meshwork as a barrier to the outflow of aqueous. Such molecules include adenyl cyclase inhibitors such as 9-(tetrahydro-2′-furyl)adenine, 2′,5′-dideoxyadenosine and miconazole, phospholipase D (PLD) activators such as roxithromycin, fluoride ion, bradykinin, progesterone, endothelin, vasopressin, 4-hydroxynonenal, interleukin-11, angiotensin II, N-(6-aminohexyl)-5-chloro-1-naphthalenesulfonamide and oxidized low density lipoprotein, cyclic adenosine monophosphate (cAMP) phosphodiesterase (CAP) activators such as phosphatidic acids such as dioleoyl, dioctanoyl and 1-stearoyl-2-arachidonyl-sn-glycerol-3-phosphate, phosphatidic acid analogues such as thiophosphatidic acid and phosphatidic acid (PA) containing alkyl ether, or vinyl ether linkages rather than ester bonds and pyrazinoyl guanidine, protein kinase A inhibitors such as (N-[2-((p-bromocinnamyl)amino)ethyl]-5-isoquinolinesulfonamide, protein phosphatase inhibitors such as vanadium salts such as potassium bisperoxo(1,10-phenanthroline)oxovanadate and dipotassium bisperoxo(picolinate)oxovanadate, molybdate oxoanions, tungstate oxoanions and dephostatins such as 3,4-dihydroxy-N-methyl-N-nitrosoaniline and 3,6-dihydroxy-N-methyl-N-nitrosoaniline, phosphatidate phosphohydrolase inhibitors/cationic amphiphiles such as propranolol, tetracaine, mepacrine, desmethylimipramine, chlopromazine and desipramine, Rho activators such as sphingosine-1-phosphate, lipid lowering agents or hyperlipoproteinemics such as 2-tetradecylglycidic acid, 5-(tetradecyloxy)-2-furoic acid, 3-thiadicarboxylic acid, 3-(4-methylpiperazin-1-yl)-1-phenylpropanone, 6,7-dihydro-5H-dibenz[c,e]azepine, N-2-n-butylindazolone, 4-phenyl-5,5-dicarbethoxy-2-pyrrolidinone, 4-(4-hydroxy-3-iodophenoxy)3,5-diiodohydrocinnamic acid, 1-methyl-4-piperidyl bis(p-chlorophenoxy)acetate, 2-[[1-methyl-2-[3-(trifluoromethyl)phenyl]ethyl]amino]ethanol benzoate ester and 5-methylpyrazinecarboxylic acid 4-oxide and concanavalin A (Con A) receptor ligands such as acetylcholinesterase and complement protein 1q.

Despite the knowledge of such compounds, a need still exists in the art to provide additional compositions and methods for the treatment of diseases termed primary open angle glaucoma.

### SUMMARY

Small organic molecules in accordance with the present invention are therapeutically useful in the treatment of diseases termed primary open angle glaucoma by having a pharmacological effect on cells and tissue. The subject small organic molecules increase the expression or enzymatic activity of MT-MMP, or a similar enzyme expressed in the TM, that activates GelA. Activation of GelA leads to increased degradation of ECM and a subsequent increase in aqueous outflow with a resultant decrease in IOP.

The carbonylic-substituted arylazine organic molecules of the present invention are also useful in establishing model systems for finding new drug therapies for diseases such as primary open angle glaucoma. To accomplish the same, the subject small organic molecules are used to modulate TM cells and tissues in vitro to effect increased expression and/or activity of MT-MMP. Evidence for this would be provided by an assay directly demonstrating increases in MT-MMP activity, which method is typically used as a primary screening tool in determining suitable compounds for use in the present invention. Alternately, such evidence could be provided by measuring the production of active species of GelA from proGelA. The activation of GelA may be demonstrated utilizing proGelA either secreted endogenously by TM cells, or added exogenously as a purified enzyme to experimental tissue or cells. In this respect it is important to note that aqueous humor has abundant proGelA. Enhancement of MT-MMP levels or activity in the cells of the outflow system of the eye would make GelA from proGelA locally available for proteolytic remodelling of ECM.

Given the importance of ECM regulation throughout the tissues and organs of the body, it is not surprising that many diseases have, as part of their pathology, an association with excessive degradation of ECM as a result of increased MMP activity. Drugs to inhibit MMP activity would be useful as therapies for diseases associated with the apparent loss of normal function and regulation of MMP. The present invention therefor includes therapeutic methods of treating particular diseases through cellular regulation of MT-MMP activity and/or expression using the small organic molecules of the present invention.

In one aspect, the present invention relates to a preparation for controlling intraocular pressure in the eye which comprises as an active compound, arylazine substituted with a carbonylic moiety. The active compound comprises an aryl group selected from homocyclic aryl and heterocyclic aryl, wherein the ring comprises at least one element selected from S, N and O.

In one form of this aspect of the invention, the active compound comprises an azinyl moiety selected from the group consisting of monoazinyl and diazinyl. Typically, the diazinyl is selected from the group consisting of 1,3-azinyl, 1,4-diazinyl and 2,3-diazinyl.

In another form of this aspect of the invention, the active compound is selected from the following general formulae [1-6], wherein 1 is derived from 1-benzazine (or quinoline), 2 is derived from 2-benzazine (or isoquinoline), 3 is derived from 1,4-benzodiazine (or quinoxaline), 4 and 5 are each derived from 1,3-benzodiazine (or quinazoline), and 6 is derived from 2,3-benzodiazine (or phthalazine): 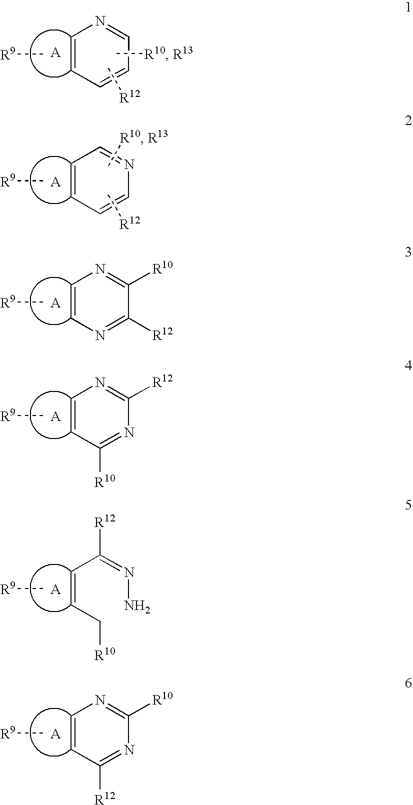
wherein:

A is a six-member homoaryl or heteroaryl ring,

R⁹ represents single or multiple non-interfering substitutions on ring A, selected from hydrido, alkyl, alkenyl, alkynyl, alkoxy, alkenoxy, hydroxy, carboxy, amino, (N-alkylcarbonyl)amino, (N-alkylcarbonyl)-N-alkylamino, (N-alkylcarbonylalkyl)amino, cyano, nitro, nitrate, arylazo, sulfo, sulfino, sulfhydryl, halo, haloalkyl, trifluoromethyl, trifluoromethylalkyl, arylalkyl, N-alkylamino, N-dialkylamino, (N-alkyl-N-alkenyl)amino, N-dialkenylamino, alkylsulfonyl, alkylsulfinyl, alkylthio, cyanoalkyl, acyl, alkylcarbonyloxy, alkenylcarbonyloxy, nitroso, alkoxyalkyl, alkoxycarbonyl, hydroxyalkyl, thiocarboxy, thiocarboxyalkyl, alkylthiocarbonyl, alkylthiocarbonylalkyl, alkoxythiocarbonyl, alkoxythiocarbonylalkyl, sulfamoyl, sulfinamoyl, N-alkylsulfamoyl, N-dialkylsulfamoyl, N-alkylsulfinamoyl, N-dialkylsulfinamoyl, sulfamoylalkyl, sulfinamoylalkyl, aminocarbonyl, aminocarbonylalkyl, N-alkylaminocarbonyl, N-dialkylaminocarbonyl, alkoxycarbonylamino, thiocarbamoyl, thiocarbamoylalkyl, (N-alkyl)thiocarbamoyl, (N-dialkyl)thiocarbamoyl, aminothio, alkylaminothio, N-dialkylaminothio, alkoxycarbonylalkyl, aminoalkyl, N-alkylaminoalkyl, N-dialkylaminoalkyl, N-alkylaminocarbonylalkyl, N-alkylaminocarbonylalkoxy; and where, if any of these substituents includes the alkyl, alkenyl or alkynyl radicals, such radicals can be straight or branched and can be from 1 to 8 carbons in length; and where the alkyl, alkenyl, or alkynyl radicals can be replaced in these substituents by C₆-C₁₅ aryl with one or two rings, cycloalkyl, cycloalkenyl, C₃-C₁₆ heteroaryl with one or two rings or heterocyclyl groups, linked via C or N; in addition, where allowed, any of these substituents including two N-dialkyl radicals and a triamine nitrogen can be replaced with a heterocyclic amine or amide;

R¹⁰ is represented by 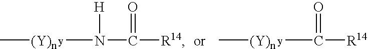
where (Y) is either (R⁴), (—O—R⁴), (—S—R⁴), or 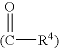
and ₙ^{y} is 0 or 1, provided that where ₙ^{y}=1, R⁴ is selected from C₁ to C₈ straight or branched alkyl, C₂ to C₈ straight or branched alkenyl, C₂ to C₈ straight or branched alkynyl, single ring C₃ to C₈ cycloalkyl, single ring C₃ to C₈ cycloalkenyl, single ring C₃ to C₈ aryl, single ring C₃ to C₈ heterocyclyl, and single ring C₃ to C₈ heteroaryl;

R¹⁴ is selected from i) —OH, —NH₂, ii) linear or branched alkoxy with C₁ to C₂₀, straight or branched alkenoxy or alkynoxy, with C₂ to C₂₀, iii) aryloxy with up to three ring systems, heteroaryloxy with 5 to 8 atoms per ring and up to three ring systems, cycloalkoxy with C₃ to C₈ and up to three ring systems, heterocycloxy with 3 to 8 atoms per ring and up to three ring systems, iv) alkylamino or dialkyl amino (—NR⁶R⁷), where R⁶ and R⁷ are as described below, and are independently, and where allowed, either hydrogen, C₁ to C₈ alkyl, C₂ to C₈ alkenyl or C₂ to C₈ alkynyl, straight or branched, v) heterocyclic or heteroaryl (—NR⁶LR⁷), where L is N, (N—N), (N—O), O, S, S(O), S(O₂), —(CH₂)—, or (═C—), where R⁶ and R⁷ are alkyl or alkenyl chains, with the total number of carbon atoms for R⁶ and R⁷ added together being between 2 and 16, and where R⁶ and R⁷ are both attached to the amido nitrogen and are cyclized via L;

R¹³ is selected from i) —OH, C₁ to C₈ alkoxy, C₂ to C₈ alkenoxy, C₂ to C₈ alkynoxy, C₆-C₁₅ aryloxy with one or two rings, cycloalkoxy, heterocycloxy, and C₃-C₁₆ heteroaryloxy with one or two rings, as well as their analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl;

ii) hydroxy C₁-C₈ alkyl, hydroxy C₂-C₈ alkenyl, hydroxy C₂-C₈ alkynyl, as well as their analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl;

and further wherein the aliphatic groups of i) and ii) are straight or branched, where allowed; and further provided that the alkyl, alkenyl, alkynyl, C₆-C₁₅ aryl with one or two rings, C₃-C₁₆ heteroaryl with one or two rings, cycloalkyl, or heterocyclyl elements of R¹⁰ and R¹³, as well as L (where L is selected from nitrogen and sulfur) can be substituted, independently and in a non-interfering manner, with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl, whose aliphatic groups are straight or branched;

R¹² represents general ring substitutions as for R⁹, and further includes

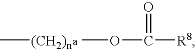

where ₙa=0, 1, or 2; and R⁸ is selected from C₁-C₈ alkyl, C₃-C₈ aryloyl alkyl, C₃-C₈ aryloyl C₃-C₈ aryl, C₃-C₈ aryl, C₁-C₈ alkylcarbonyl C₃-C₈ aryl, C₁-C₈ alkoxy carbonyl C₃-C₈ aryl, and C₁-C₈ alkoxy carbonyl C₁-C₈ alkyl; and further providing that alkenyl or alkynyl can be substituted for alkyl in R⁸, heteroaryl can be substituted for aryl in R⁸, provided that the aliphatic groups can be straight or branched; as well as analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl, whose aliphatic groups can be straight or branched.

In still another form of this aspect of the invention, for A, the homoaryl is phenyl, the heteroaryl is pyridinyl, and the halo is selected from fluoro, chloro, bromo and iodo. Preferably, at least one of the halo C₁-C₈ alkyl groups is trifluoromethyl.

In one embodiment of this aspect of the invention, the active compound is a compound of formula 1, kynurenic acid, also known as hydroxyquinoline carboxylic acid, or 4-hydroxy-2-quinoline carboxylic acid, or 4-hydroxyquinaldic acid.

In another embodiment of this aspect of the invention, the active compound is a compound of formula 2.

In another embodiment of this aspect of the invention, the active compound is a compound of formula 3.

In another embodiment of this aspect of the invention, the active compound is a compound of formula 4.

In another embodiment of this aspect of the invention, the active compound is a compound of formula 5.

In another embodiment of this aspect of the invention, the active compound is a compound of formula 6.

In another form of this aspect of the invention, the active compound is a derivative of kynurenic acid. Typically, the derivative of kynurenic acid is selected from the group consisting of 5,7-dichlorokynurenic acid and 3-hydroxy-2-methyl4-quinoline carboxylic acid.

In still another form of this aspect of the invention, the preparation further comprises a counterion for balancing the active compound, said counterion being selected from:
a) A′-L₁-NH-L₃-NH-L₂-B, wherein

L₁ and L₂ are independently selected from methylene, ethylene, propylene, isopropylene, and cyclopropylene;

L₃ is alkyl C₁-C₆, linear or branched, and can be replaced in part or entirety with cycloalkyl C₃-C₆, or the alkyl component of L₃ can be substituted with cycloalkyl C₃-C₆ in a spiro configuration such that the maximum total number of carbon atoms in L₃ is 6; and A′ and B are independently phenyl, naphthyl, or heteroaryl;
b) A′-L₁-NH-L₂-B, wherein A′, B, L₁, L₂ are as described above; 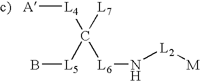
   wherein

A′, B, and M are independently phenyl, naphthyl, or heteroaryl;

L₂ is as described above;

L₄, L₅, L₆ are independently Cₙ, where n=0, 1, or 2;

L₇ is selected from —H, alkyl C₁-C₆, linear or branched which may be replaced, in part or in its entirety, with cycloalkyl C₃-C₆; 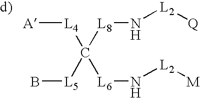
wherein

A′, B, M, and Q are independently phenyl, naphthyl, or heteroaryl;

L₂ is as described above;

L₄, L₅, L₆, L₈ are independently Cₙ, where n =0, 1, or 2; further providing that for a) through d) above:

1) where allowed, the hydrogens on all alkyl or cycloalkyl groups can be substituted with i) straight or branched C₁-C₆ alkyl, C₂-C₈ alkenyl, C₂-C₈ alkynyl, C₃-C₈ cycloalkyl, C₃-C₈ cycloalkenyl, or combinations thereof; or ii) straight or branched C₁-C₆ alkoxy, C₂-C₈ alkenoxy, C₂-C₈ alkynoxy, or C₃-C₈ cycloalkoxy, whose aliphatic groups are straight or branched;

2) where allowed, the hydrogens on all aromatic rings can be substituted with a group selected from i) C₁-C₈ alkyl, C₂-C₈ alkenyl, C₂-C₈ alkynyl, C₃-C₈ cycloalkyl, C₃-C₈ cycloalkenyl, C₁-C₈ alkoxy, C₂-C₈ alkenoxy, C₂-C₈ alkynoxy, C₂-C₈ thioalkyl, C₂-C₈ thioalkenyl, and C₂-C₈ thioalkynyl, whose aliphatic groups are straight or branched; and ii) hydroxy, halo, nitro, cyano, and halomethyl; and

3) all optical isomers are permitted.

The counterion can be benzathine (Ph-CH₂—NH—(CH₂)₂—NH—CH₂-Ph, where Ph=phenyl), either as a base or as a salt with an acceptable anion.

In an embodiment of this form of the invention, the preparation comprises kynurenic acid and/or a derivative of kynurenic acid as the active compound.

In another embodiment of this form of the invention, the anion is selected from chloride, propionate, and acetate.

In another embodiment of this form of the invention, the molar ratio of active compound to counterion is no greater than about 3:1, say, between about 1:1 and about 2:1.

In another form of this aspect of the invention, the active compound is useful to increase the activity of Gelatinase A in ocular cells. Preferably, the active compound is selected from kynurenic acid and derivatives of kynurenic acid.

In another aspect, the present invention relates to a pharmaceutically acceptable composition comprising: any of the above preparations containing the active compound in a therapeutically effective amount to increase the activity of Gelatinase A in ocular cells.

In yet another aspect, the present invention relates to a method of administering an above-described pharmaceutically acceptable composition comprising: formulating the composition as a sterile aqueous or non-aqueous solution; and applying the solution on or within an eye.

In still another aspect, the present invention relates to a method of administering an above-described pharmaceutically acceptable composition comprising: providing the composition in the form of an ocular implant; and implanting the ocular implant within an eye. Typically, the implant can be selected from a biodegradable matrix and a drug-eluting reservoir.

In yet still another embodiment, the present invention relates to a method of administering an above-described pharmaceutically acceptable composition: formulating the composition as a sterile ointment; and applying the ointment on or near an eye.

In still another aspect, the present invention relates to a method of administering an above-described pharmaceutically acceptable composition comprising: formulating said composition as a sterile gel; and applying said gel on or near an eye.

In yet another aspect, the present invention relates to a method of administering an above-described pharmaceutically acceptable composition comprising: providing the composition combined with biodegradable polymer matrix, as particles whose largest dimension is less than 10 microns; and applying the particles on or near an eye.

### DETAILED DESCRIPTION OF THE INVENTION

The following detailed description is provided to enable any person skilled in the art to which the present invention pertains to make and use the same, and sets forth the best mode contemplated by the inventors of carrying out the subject invention.

The present invention is the use of small organic molecules that have a pharmacological effect on cells and tissues to increase the enzymatic activity and/or expression of one or more membrane-type matrix metalloproteinases (MT-MMPs), or a similar enzyme, expressed in the trabecular meshwork (TM) of an eye to activate Gelatinase A (GelA) for the treatment of primary open angle glaucoma.

The use of the subject small organic molecules of the present invention for increasing cell membrane expression of MT-MMPs, and as a result, for activating GelA, increases the turnover and reduces the accumulation of extracellular matrix. Activating GelA in the TM increases outflow of aqueous and lowers intraocular pressure, thereby having therapeutic potential in the treatment of primary open angle glaucoma.

An additional use of the subject small organic molecules of the present invention is for increasing cell membrane expression of one or more MT-MMPs in the development of in vitro models, which could be used in the discovery of new medical treatments. In general, the small organic molecules of the present invention could contribute to the discovery of new treatments for any ocular disease with a pathophysiology involving changes in expression of one or more MT-MMPs and activation of GelA.

The invention contemplates all such isomers both individually, in pure form and in admixture, including racemic mixtures.

The active compounds of the present invention are described in still greater detail in the examples that follow.

### EXAMPLE

### Carbonylic-Substituted Aryl Azines that Increase Expression or Activity of a Cell-Associated Component with Gelatinase Activity, Detected Using a Cell-Based Screening Assay that Measures Hydrolysis of a Thiopeptolide Substrate

A. Thiopeptolide Assay Used to Detect Cell-Associated Gelatinase Activity Ascribed to MT-MMP:

Rhesus monkey TM cells are cultured and maintained for at least two weeks in 96-well microtiter plates in a growth medium such as Dulbecco's Modified Eagles' Medium (DMEM) plus 15 percent (v/v) fetal bovine serum (FBS) containing 1% bovine calf serum or a medium more suitable for endothelial cells due to its lower serum content such as MCDB 131 supplemented with endothelial cell growth supplement, 1% or less FBS or bovine calf serum and defined supplements as described by Knedler and Ham, In Vitro Cellular and Dev. Biol. 23:481 (1987). Two days before molecule testing, the medium is replaced by a defined, serum-free medium such as Minimum Essential Medium (MEM) containing defined supplements as described by Schachtschabel and Binninger, Z.f. Gerontol. 26:243 (1993), but preferably using a basal medium such as MCDB 131 containing defined supplements, because of the absence of interfering substances that could bind or compete with test compounds and because of its ability to maintain endothelial-like cells such as TM cells as a stable, nonproliferative monolayer while optimizing expression of native structural and functional attributes. Unless specifically noted, all molecules tested were prepared as stock solutions in dimethyl sulfoxide (DMSO) with a final concentration of 10 mg/ml. The stocks were stored in a dessicator at −20 degrees Celsius. In testing the molecules, the molecules were diluted to final concentrations of 0.3 ug/ml and 15 ug/ml in a simplified culture medium based on Ames' medium, called Concanavalin A (Con A) conditioning medium (CACM). The monkey TM cells were incubated with the test molecules for 48 hours.

Control medium of CACM plus DMSO and a positive control with 5 ug/ml Con A plus DMSO were run in parallel. At the end of the incubation period, the experimental media were replaced with 100 uL of the buffer that is part of the thiopeptolide assay mixture (50 mmol/L HEPES, 5 mmol/L CaCl₂, 3.5 mmol/L KCl, 106 mmol/L NaCl, 0.02% (v/v) Brij 35, pH 7.5). Next 100 uL of a doubly (2×) concentrated mixture of the thiopeptolide substrate (from a forty times (40×) concentrated DMSO stock, to give 1 mmol/L final concentration) freshly combined with the thiol reagent (5,5′dithiobis(2-nitrobenzoic acid (DTNB) from a twenty times (20×) concentrated DMSO stock to give 1 mmol/L) were added to each well and were incubated at 37 degrees Celsius for two hours with gentle agitation.

At the end of the incubation period, by means of a spectrophoto-metric plate reader, the optical density (OD) at 410 nm was determined for each well after automatic subtraction of a blank value for a well containing reaction mixture but without cells. The average OD at the two-hour end point for each test molecule at all concentrations in triplicate was calculated. This calculation was interpreted to be a measure of cell-associated MT-MMP level, equivalently defined as either its activity (a catalytic property of the enzyme) or expression (number of functional molecules). The percent difference in OD for each sample compared to the CACM control reflects the effectiveness of each test molecule or combination of molecules in eliciting increases in MT-MMP levels. As an alternate to the end point OD reading, one may use the mean rate of appearance of the reaction product (mean V), calculated from the best linear fit of the data to absorbance vs. time. In so doing, measurements are taken every five minutes with the first and possibly last time points, that do not contribute to a good linear fit, routinely eliminated.

B. Testing of Small Organic Molecules for Associated MT-MMP Activity:

TM cells were exposed to molecules and combinations of molecules by means of simultaneous addition, from separate stocks, to incubation medium.

1. Group 1 Molecule: Kynurenic Acid

Kynurenic acid obtained from Sigma-Aldrich of St. Louis, Mo. USA, was tested for MT-MMP activity according to the procedure set out above.

Results are set forth below in Table 1.

TABLE 1

| Compound | Conc. | Benz. Conc. | Activity |
|---|---|---|---|
| Kynurenic Acid | 1.6 | 0.0 | 11.4 |
| ″ | 79.3 | 0.0 | 23.2 |
| ″ | 1.6 | 0.83 | 12.9 |
| | 79.3 | 41.6 | 297.1 |

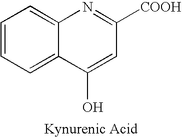

Group 1 Molecule: Kynurenic Acid with Benzathine Counterion

Benzathine (as diacetate salt) obtained from Fluka (Sigma-Aldrich) of St. Louis, Mo., USA, is combined with an equal weight of kynurenic acid by simultaneous addition to the above incubation medium and evince MT-MMP activity when tested according to the procedure set out above. For the purposes of consistency and ease of handling, the concentration of the test compounds matches that of benzathine in weight percent, as opposed to maintaining integral molar ratios. Activity displays a dose dependency relative to the concentration of the test compound. In the primary assay, substantial activity over control levels was only seen at the 15 ug/ml dose of test compound.

There was a consistent increase in the activity of these compounds when benzathine was added in the incubation mixture applied to TM cells.

While DMSO was the solvent most commonly used for concentrated stocks, the use of aqueous stocks for a few compounds, in combination with benzathine, also from an aqueous stock, did not impair the ability of these compounds to generate significant increases in MT-MMP expression.

A convenient form for administering one or more organic molecules of the present invention to increase Gelatinase A activity in ocular cells is through a pharmaceutically acceptable composition comprising one or more of the following: one or more organic molecules or one or more hydrates of the molecules; one or more organic molecules or one or more acid addition salts of the molecules whereby suitable acids include for example but are not limited to mineral acids such as hydrohalic acids, organic acids such as acetic acid, or acids which are sparingly soluble and impart slow-release properties to their salts, such as pamoic acid; and one or more organic molecules or one or more base addition salts of the molecules whereby suitable salts include those formed from inorganic bases such as hydroxides, carbonates, bicarbonates, or alkoxides of the alkali or alkaline earth metals, organic bases such as mono-, di-, and trialkylamines, alkanolamines, alkene-diamines, phenylalkylamines, cyclic saturated bases, cyclic unsaturated bases or alkylamines forming quaternary salts.

The organic bases forming such salts are of suitable molecular size to be therapeutically acceptable. Acid and base addition salts in accordance with the present invention are prepared by conventional means known by those skilled in the art.

If such pharmaceutically acceptable compositions are formulated as a sterile solution or a suspension in water or other aqueous media, the above formulation would likewise include physiological salt solutions whereby the pH is suitably adjusted and/or buffered and the tonicity is suitably adjusted for optimal absorption, distribution, release, and/or efficacy at the site of action on or within the eye.

If such pharmaceutically acceptable compositions are formulated as a non-aqueous solution or suspension, the above formulation would likewise include an oil, an organic solvent or methyl sulfoxide. Also, formulations of the present invention could likewise include cyclodextrin, a detergent or other non-toxic pharmaceutical excipients combined covalently or noncovalently with a biodegradable or a nonerodable encapsulating substance such as a polymer, as known to those skilled in the art.

The organic compounds of the present invention are administered to treat glaucoma through a method of delivery to the tissues of the trabecular meshwork of the eye. Methods of such delivery of one or more of the organic molecules of the present invention include for example but are not limited to application of externally applied eye drops, ointments or implants, injection or insertion a solution, suspension, or sustained-release implant into the anterior chamber or sclera of an eye, external application on the scleral surface of an eye and/or administration as an adjunct pharmaceutical treatment at the time of surgical treatment for glaucoma, as with filtration surgery. Various forms of delivery include single or multiple dosages such that an acute, short term therapy schedule performed once or intermittently over a specified time frame could be useful as an alternative to sustained therapy. In this way, the compounds could be useful for effecting varying degrees of amplification of aqueous outflow through the trabecular meshwork and adjoining structures.

While there are described herein certain specific embodiments of the present invention, it will be manifest to those skilled in the art that various modifications may be made without departing from the spirit and scopeof the underlying inventive concept and that the same is not limited to the particular forms herein described except insofar as indicated by the scope of the appended claims.

We claim:

1. A preparation for controlling intraocular pressure in the eye that comprises as an active compound, arylazine substituted with a carbonylic moiety.

2. The preparation of claim 1 wherein said active compound comprises an aryl group selected from homocyclic aryl and heterocyclic aryl.

3. The preparation of claim 1 wherein said active compound comprises an azinyl moiety selected from the group consisting of monoazinyl and diazinyl.

4. The preparation of claim 3 wherein said diazinyl is selected from the group consisting of 1,3-azinyl, 1,4-diazinyl and 2,3-diazinyl.

5. The preparation of claim 1 wherein said active compound is selected from the following general formulae [1-6]: 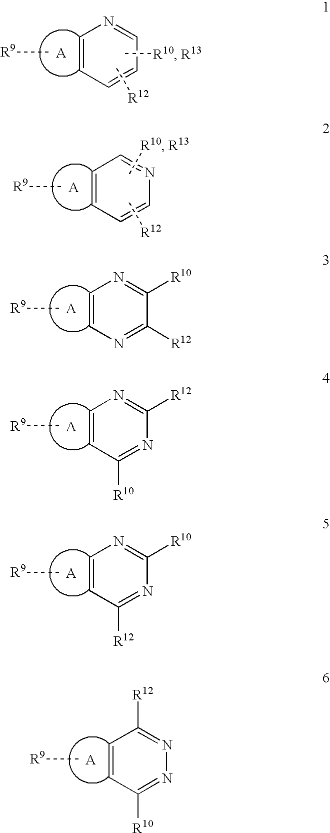
wherein:
A is a six-member homoaryl or heteroaryl ring,
R⁹ represents single or multiple non-interfering substitutions on ring A, selected from hydrido, alkyl, alkenyl, alkynyl, alkoxy, alkenoxy, hydroxy, carboxy, amino, (N-alkylcarbonyl)amino, (N-alkylcarbonyl)-N-alkylamino, (N-alkylcarbonylalkyl)amino, cyano, nitro, nitrate, arylazo, sulfo, sulfino, sulfhydryl, halo, haloalkyl, trifluoromethyl, trifluoromethylalkyl, arylalkyl, N-alkylamino, N-dialkylamino, (N-alkyl-N-alkenyl)amino, N-dialkenylamino, alkylsulfonyl, alkylsulfinyl, alkylthio, cyanoalkyl, acyl, alkylcarbonyloxy, alkenylcarbonyloxy, nitroso, alkoxyalkyl, alkoxycarbonyl, hydroxyalkyl, thiocarboxy, thiocarboxyalkyl, alkylthiocarbonyl, alkylthiocarbonylalkyl, alkoxythiocarbonyl, alkoxythiocarbonylalkyl, sulfamoyl, sulfinamoyl, N-alkylsulfamoyl, N-dialkylsulfamoyl, N-alkylsulfinamoyl, N-dialkylsulfinamoyl, sulfamoylalkyl, sulfinamoylalkyl, aminocarbonyl, aminocarbonylalkyl, N-alkylaminocarbonyl, N-dialkylaminocarbonyl, alkoxycarbonylamino, thiocarbamoyl, thiocarbamoylalkyl, (N-alkyl)thiocarbamoyl, (N-dialkyl)thiocarbamoyl, aminothio, alkylaminothio, N-dialkylaminothio, alkoxycarbonylalkyl, aminoalkyl, N-alkylaminoalkyl, N-dialkylaminoalkyl, N-alkylaminocarbonylalkyl, N-alkylaminocarbonylalkoxy; and where, if any of these substituents includes the alkyl, alkenyl or alkynyl radicals, such radicals can be straight or branched and can be from 1 to 8 carbons in length; and where the alkyl, alkenyl, or alkynyl radicals can be replaced in these substituents with C₆-C₁₅ aryl with one or two rings, cycloalkyl, cycloalkenyl, C₃-C₁₆ heteroaryl with one or two rings or heterocyclyl groups, linked via C or N; in addition, where allowed, any of these substituents including two N-dialkyl radicals and a triamine nitrogen can be replaced with a heterocyclic amine or amide;
R¹⁰ is represented by 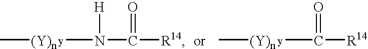
where (Y) is either (R⁴), (—O—R⁴), (—S—R⁴), or 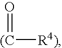
and ₙ^{y} is 0 or 1, provided that where ₙ^{y}=1, R⁴ is selected from C₁ to C₈ straight or branched alkyl, C₂ to C₈ straight or branched alkenyl, C₂ to C₈ straight or branched alkynyl, single ring C₃ to C₈ cycloalkyl, single ring C₃ to C₈ cycloalkenyl, single ring C₃ to C₈ aryl, single ring C₃ to C₈ heterocyclyl, and single ring C₃ to C₈ heteroaryl;
R¹⁴ is selected from i) —OH, —NH₂, ii) linear or branched alkoxy with C₁ to C₂₀, straight or branched alkenoxy or alkynoxy, with C₂ to C₂₀, iii) aryloxy with up to three ring systems, heteroaryloxy with 5 to 8 atoms per ring and up to three ring systems, cycloalkoxy with C₃ to C₈ and up to three ring systems, heterocycloxy with 3 to 8 atoms per ring and up to three ring systems, iv) alkylamino or dialkyl amino (—NR⁶R⁷), where R⁶ and R⁷ are as described below, and are independently, and where allowed, either hydrogen, C₁ to C₈ alkyl , C₂ to C₈ alkenyl or C₂ to C₈ alkynyl, straight or branched, v) heterocyclic or heteroaryl (—NR⁶LR⁷), where L is N, (N—N), (N—O), O, S, S(O), S(O₂), —(CH₂)—, or (═C—), where R⁶ and R⁷ are alkyl or alkenyl chains, with the total number of carbon atoms for R⁶ and R⁷ added together being between 2 and 16, and where R⁶ and R⁷ are both attached to the amido nitrogen and are cyclized via L;
R¹³ is selected from i) —OH, C₁ to C₈ alkoxy, C₂ to C₈ alkenoxy, C₂ to C₈ alkynoxy, C₆-C₁₅ aryloxy with one or two rings, cycloalkoxy, heterocycloxy, and C₃-C₁₆ heteroaryloxy with one or two rings, as well as their analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl;
ii) hydroxy C₁-C₈ alkyl, hydroxy C₂-C₈ alkenyl, hydroxy C₂-C₈ alkynyl, as well as their analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl;
and further wherein the aliphatic groups of i) and ii) are straight or branched, where allowed; and further provided that the alkyl, alkenyl, alkynyl, C₆-C₁₅ aryl with one or two rings, C₃-C₁₆ heteroaryl with one or two rings, cycloalkyl, or heterocyclyl elements of R¹⁰ and R¹³, as well as L (where L is selected from nitrogen and sulfur) can be substituted, independently and in a non-interfering manner, with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl, whose aliphatic groups are straight or branched;
R¹² represents general ring substitutions as for R⁹, and further includes 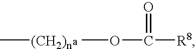
where ₙa=0, 1, or 2; and R⁸ is selected from C₁-C₈ alkyl, C₃-C₈ aryloyl alkyl, C₃-C₈ aryloyl C₃-C₈ aryl, C₃-C₈ aryl, C₁-C₈ alkylcarbonyl C₃-C₈ aryl, C₁-C₈ alkoxy carbonyl C₃-C₈ aryl, and C₁-C₈ alkoxy carbonyl C₁-C₈ alkyl; and further providing that alkenyl or alkynyl can be substituted for alkyl in R⁸, heteroaryl can be substituted for aryl in R⁸, provided that the aliphatic groups can be straight or branched; as well as analogues substituted with at least one substituent selected from the group consisting of hydroxy, halo, C₁-C₈ alkyl, C₂-C₈ alkenyl, C₂-C₈ alkynyl, nitro, cyano, sulfo, sulfino, amino, cycloalkyl having a ring of 3 to 8 carbons, cycloalkoxy having a ring of 3 to 8 carbons, carboxyl C₁-C₈ alkyl, hydroxythiocarbonyl, hydroxythiocarbonyl C₁-C₈ alkyl, acyl, C₁-C₈ alkylamino, C₁-C₈ alkoxy, C₂-C₈ alkenoxy, C₂-C₈ alkynoxy, C₁-C₈ alkylthio, dialkylamino with the total number of carbon atoms added together being between 2 and 16, aminosulfonyl, and halo C₁-C₈ alkyl, whose aliphatic groups can be straight or branched.

6. The preparation of claim 5 wherein for A, said homoaryl is phenyl, said heteroaryl is pyridinyl, said halo is selected from fluoro, chloro, bromo and iodo.

7. The preparation of claim 5 wherein said halo C₁-C₈ alkyl is trifluoromethyl.

8. The preparation of claim 5 wherein said active compound is kynurenic acid (hydroxyquinoline carboxylic acid).

9. The preparation of claim 5 wherein said active compound is a derivative of kynurenic acid.

10. The preparation of claim 9 wherein said active compound is selected from the group consisting of 5,7-dichloro kynurenic acid and 3-hydroxy-2-methyl4-quinoline carboxylic acid.

11. The preparation of claim 5 which further comprises a counterion for balancing said active compound, said counterion being selected from:
a) A′-L₁-NH -L₃-NH-L₂-B, wherein
L₁ and L₂ are independently selected from methylene, ethylene, propylene, isopropylene, and cyclopropylene;
L₃ is alkyl C₁-C₆, linear or branched, and can be replaced in part or entirety with cycloalkyl C₃-C₆, or the alkyl component of L₃ can be substituted with cycloalkyl C₃-C₆ in a spiro configuration such that the maximum total number of carbon atoms in L₃ is 6; and A′ and B are independently phenyl, naphthyl, or heteroaryl;
b) A′-L₁-NH-L₂-B, wherein A′, B, L₁, L₂ are as described above; 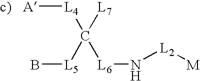
wherein
A′, B, and M are independently phenyl, naphthyl, or heteroaryl;
L₂ is as described above;
L₄, L₅, L₆ are independently Cₙ, where n=0, 1, or 2;
L₇ is selected from —H, alkyl C₁-C₆, linear or branched which may be replaced, in part or in its entirety, with cycloalkyl C₃-C₆; 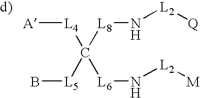
wherein
A′, B, M, and Q are independently phenyl, naphthyl, or heteroaryl;
L₂ is as described above;
L₄, L₅, L₆, L₈ are independently Cₙ, where n=0, 1, or 2; further providing that for a) through d) above:
1) where allowed, the hydrogens on all alkyl or cycloalkyl groups can be substituted with i) straight or branched C₁-C₆ alkyl, C₂-C₈ alkenyl, C₂-C₈ alkynyl, C₃-C₈ cycloalkyl, C₃-C₈ cycloalkenyl, or combinations thereof; or ii) straight or branched C₁-C₆ alkoxy, C₂-C₈ alkenoxy, C₂-C₈ alkynoxy, or C₃-C₈ cycloalkoxy, whose aliphatic groups are straight or branched;
2) where allowed, the hydrogens on all aromatic rings can be substituted with a group selected from i) C₁-C₈ alkyl, C₂-C₈ alkenyl, C₂-C₈ alkynyl, C₃-C₈ cycloalkyl, C₃-C₈ cycloalkenyl, C₁-C₈ alkoxy, C₂-C₈ alkenoxy, C₂-C₈ alkynoxy, C₂-C₈ thioalkyl, C₂-C₈ thioalkenyl, and C₂-C₈ thioalkynyl, whose aliphatic groups are straight or branched; and ii) hydroxy, halo, nitro, cyano, and halomethyl; and
3) all optical isomers are permitted.

12. The preparation of claim 11 wherein said counterion is benzathine (Ph-CH₂—NH—(CH₂)₂—NH—CH₂-Ph, where Ph=phenyl), either as a base or as a salt with an acceptable anion.

13. The preparation of claim 12 wherein said active compound is kynurenic acid.

14. The preparation of claim 12 wherein said active compound is a derivative of kynurenic acid.

15. The preparation of claim 12 wherein said anion is selected from chloride, propionate, and acetate.

16. The preparation of claim 11 wherein the molar ratio of active compound to counterion is no greater than about 3:1.

17. The preparation of claim 16 wherein the molar ratio of active compound to counterion ranges between about 1:1 and about 2:1.

18. The preparation of claim 5 wherein said active compound is useful to increase the activity of Gelatinase A in ocular cells.

19. The preparation of claim 18 wherein said active compound is selected from kynurenic acid.

20. The preparation of claim 18 wherein said active compound is selected from a derivative of kynurenic acid.

21. The preparation of claim 5 wherein said active compound is useful to increase the activity of Gelatinase A in ocular cells.

22. The preparation of claim 11 wherein said active compound is useful to increase the activity of Gelatinase A in ocular cells.

23. The preparation of claim 13 wherein said active compound is useful to increase the activity of Gelatinase A in ocular cells.

24. A pharmaceutically acceptable composition comprising: the preparation of claim 5 containing said active compound in a therapeutically effective amount to increase the activity of Gelatinase A in ocular cells.

25. A pharmaceutically acceptable composition comprising: the preparation of claim 11 containing said active compound in a therapeutically effective amount to increase the activity of Gelatinase A in ocular cells.

26. A pharmaceutically acceptable composition comprising: the preparation of claim 13 containing said active compound in a therapeutically effective amount to increase the activity of Gelatinase A in ocular cells.

27. A method of administering the pharmaceutically acceptable composition of claim 24 comprising:
formulating said composition as a sterile aqueous or non-aqueous solution; and
applying said solution on or within an eye.

28. A method of administering the pharmaceutically acceptable composition of claim 25 comprising:
formulating said composition as a sterile aqueous or non-aqueous solution; and
applying said solution on or within an eye.

29. A method of administering the pharmaceutically acceptable composition of claim 26 comprising:
formulating said composition as a sterile aqueous or non-aqueous solution; and
applying said solution on or within an eye.

30. A method of administering the pharmaceutically acceptable composition of claim 24 comprising:
providing said composition in the form of an ocular implant; and
implanting said ocular implant within an eye.

31. The method of claim 30 wherein said implant comprises a biodegradable matrix.

32. The method of claim 30 wherein said implant comprises a drug-eluting reservoir.

33. A method of administering the pharmaceutically acceptable composition of claim 25 comprising:
providing said composition in the form of an ocular implant; and
implanting said ocular implant within an eye.

34. The method of claim 33 wherein said implant comprises a biodegradable matrix.

35. The method of claim 33 wherein said implant comprises a drug-eluting reservoir.

36. A method of administering the pharmaceutically acceptable composition of claim 26 comprising:
providing said composition in the form of an ocular implant; and
implanting said ocular implant within an eye.

37. The method of claim 36 wherein said implant comprises a biodegradable matrix.

38. The method of claim 36 wherein said implant comprises a drug-eluting reservoir.

39. A method of administering the pharmaceutically acceptable composition of claim 24 comprising:
formulating said composition as a sterile ointment; and
applying said ointment on or near an eye.

40. A method of administering the pharmaceutically acceptable composition of claim 25 comprising:
formulating said composition as a sterile ointment; and
applying said ointment on or near an eye.

41. A method of administering the pharmaceutically acceptable composition of claim 26 comprising:
formulating said composition as a sterile ointment; and
applying said ointment on or near an eye.

42. A method of administering the pharmaceutically acceptable composition of claim 24 comprising:
formulating said composition as a sterile gel; and
applying said gel on or near an eye.

43. A method of administering the pharmaceutically acceptable composition of claim 25 comprising:
formulating said composition as a sterile gel; and
applying said gel on or near an eye.

44. A method of administering the pharmaceutically acceptable composition of claim 26 comprising:
formulating said composition as a sterile gel; and
applying said gel on or near an eye.

45. A method of administering the pharmaceutically acceptable composition of claim 24 comprising:
providing said composition combined with biodegradable polymer matrix, as particles whose largest dimension is less than 10 microns; and
applying said particles on or near an eye.

46. A method of administering the pharmaceutically acceptable composition of claim 25 comprising:
providing said composition combined with biodegradable polymer matrix, as particles whose largest dimension is less than 10 microns; and
applying said particles on or near an eye.

47. A method of administering the pharmaceutically acceptable composition of claim 26 comprising:
providing said composition combined with biodegradable polymer matrix, as particles whose largest dimension is less than 10 microns; and
applying said particles on or near an eye.
